(19) United States
(12) Patent Application Publication
(10) Pub. No.: US 2010/0098632 A1
(43) Pub. Date: Apr. 22, 2010

(54) HYDROXYAPATITE PARTICLES

(76) Inventors: Stephen J. Russell, Rochester, MN (US); Kah-Whye Peng, Rochester, MN (US); Hooi Tin Ong, Rochester, MN (US)

Correspondence Address: FISH & RICHARDSON P.C., PO BOX 1022, MINNEAPOLIS, MN, 55440-1022, US

(21) Appl. No.: 12/373,485

(22) PCT Filed: Jul. 12, 2007
(86) PCT No.: PCT/US07/73397

§ 371 Date: Oct. 16, 2009

Related U.S. Application Data
(60) Provisional application No. 60/830,433, filed on Jul. 12, 2006.

(51) Int. Cl. A61K 51/04; C07F 5/00; C07K 16/00; A61P 35/00; A61K 31/663

(52) U.S. Cl. 424/1.77; 534/10; 562/13; 562/21; 530/391.3; 514/108

(57) ABSTRACT

This document provides methods and materials related to hydroxyapatite particles. For example, hydroxyapatite particles, methods for making hydroxyapatite particles, and methods for using hydroxyapatite particles are provided herein.

### BACKGROUND

1. Technical Field

This document relates to hydroxyapatite particles. For example, this document relates to radiolabeled hydroxyapatite particles as well as methods for making and using such hydroxyapatite particles.

2. Background Information

Hydroxyapatite particles are small particles that have been used as implant materials. For example, hydroxyapatite particles have been used in bone replacement and in dental applications such as alveolar ridge augmentations, root extraction site fillings, and restoration of periodontal osseous lesions.

### SUMMARY

This document provides methods and materials related to hydroxyapatite particles. For example, this document provides hydroxyapatite particles, methods for making hydroxyapatite particles, and methods for using hydroxyapatite particles. The hydroxyapatite particles provided herein can have a radionuclide such as a radionuclide attached to a biphosphonate. Such hydroxyapatite particles can have an increased stability such that the radioactivity leaches at a rate less than 50 percent per hour. In some cases, a hydroxyapatite particle provided herein can contain radioactivity that does not exhibit detectable leaching for up to 6 hours, 12 hours, 18 hours, 1 day, 2 days, 3 days, 4 days, or 5 days.

In some cases, the hydroxyapatite particles provided herein can contain a binding molecule such as an antibody, receptor ligand, or nucleic acid. The binding molecule of such hydroxyapatite particles can be used to target the hydroxyapatite particles and radioactivity to a particular location within a mammal's body. For example, anti-CD46 antibodies can be attached to hydroxyapatite particles such that the hydroxyapatite particles are targeted to CD46 positive cells such as tumor cells.

The hydroxyapatite particles provided herein can be used to image particular tissues within a mammal's body, to deliver therapeutic agents (e.g., a drug attached to the hydroxyapatite particles) to particular tissues within a mammal's body, and to deliver therapeutic doses of radiation to particular tissues within a mammal's body.

This document also provides methods for making hydroxyapatite particles. For example, the hydroxyapatite particles provided herein can be made using a synthesis method that comprises adding biphosphonate-conjugated radionuclides during the synthesis of the hydroxyapatite particles. The resulting radioactive hydroxyapatite particles can be stable. In some case, binding molecules such as monoclonal antibodies can be added to the synthesis reaction such that the resulting radioactive hydroxyapatite particles can be targeted to, for example, specific cell surface receptors.

In general, one aspect of this document features an article of manufacture comprising, or consisting essentially of, hydroxyapatite particles comprising a radionuclide attached to a biphosphonate, wherein the average diameter of the hydroxyapatite particles is between 40 nm and 200 nm. The radionuclide can comprise Sm-153, Tc-99m, ¹²³I, ¹⁸F, ¹³¹I, ¹¹¹In, ¹⁸⁸Re, ¹⁶⁶Ho, ⁹⁰Y, or ⁸²Rb. The biphosphonate can be ethylene diamine tetramethylene phosphoric acid or methylene diphosphonate. The average diameter of the hydroxyapatite particles can be between 60 nm and 200 nm. The hydroxyapatite particles can comprise a binding molecule. The binding molecule can be an antibody. The antibody can be an anti-CD46 antibody, an anti-CD20 antibody, an anti-CD38 antibody, an anti-Her-2 antibody, an anti-EGFR antibody, an anti-α folate receptor antibody, an anti-MOV18 antibody, or an anti-MOV19 antibody. The binding molecule can be a receptor ligand. The particles can comprise a nucleic acid molecule.

In another aspect, this document features a method for making a radioactive hydroxyapatite particle. The method can comprise, or consist essentially of, synthesizing a hydroxyapatite particle in the presence of a radionuclide attached to a biphosphonate. The radionuclide can comprise Sm-153, Tc-99m, ¹²³I, ¹⁸F, ¹³¹I, ¹¹¹In, ¹⁸⁸Re, ¹⁶⁶Ho, ⁹⁰Y, or ⁸²Rb. The biphosphonate can be ethylene diamine tetramethylene phosphoric acid or methylene diphosphonate. The average diameter of the hydroxyapatite particles can be between 40 nm and 200 nm. The hydroxyapatite particles can comprise a binding molecule. The binding molecule can be an antibody. The antibody can be an anti-CD46 antibody, an anti-CD20 antibody, an anti-CD38 antibody, an anti-Her-2 antibody, an anti-EGFR antibody, an anti-α folate receptor antibody, an anti-MOV18 antibody, or an anti-MOV19 antibody. The binding molecule can be a receptor ligand. The hydroxyapatite particle can comprise a nucleic acid molecule.

In another aspect, this document features a method for treating a mammal having cancer. The method comprises, or consists essentially of, administering to the mammal hydroxyapatite particles comprising a radionuclide attached to a biphosphonate under conditions where the progression rate of the cancer is reduced. The mammal can be a human. The cancer can be selected from the group consisting of liver cancer, spleen cancer, and kidney cancer. The biphosphonate can be ethylene diamine tetramethylene phosphoric acid or methylene diphosphonate. The radionuclide can comprise Sm-153, Tc-99m, ¹²³I, ¹⁸F, ¹³¹I, ¹¹¹In, ¹⁸⁸Re, ¹⁶⁶Ho, ⁹⁰Y, or ⁸²Rb. The hydroxyapatite particles can comprise a binding molecule. The hydroxyapatite particles can comprise a therapeutic agent. The therapeutic agent can be a chemotherapeutic agent or a phosphonate.

In another aspect, this document features a method of depleting Kupffer cells in a mammal. The method comprises, or consists essentially of, administering to the mammal hydroxyapatite particles comprising a phosphonate under conditions where the number of Kupffer cells in the liver of the mammal is reduced. The mammal can be a human. The phosphonate can be clodronate.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains photographs of the distribution of Sm-HAP uptake in mice. Strong signals were observed in liver and spleen via non-invasive gamma camera imaging using an X-SPECT (Gamma Medica, CA) machine. The mouse was injected intravenously with Sm-153 HA nanoparticles synthesized at 50° C. Imaging was performed 2 hours post injection.

FIG. 2 contains a photograph of the distribution of Sm-153 EDTMP determined via non-invasive imaging using a small animal gamma camera (X-SPECT).

FIG. 3 contains gamma camera images of a mouse intravenously given 500 μCi of Sm-HAP tagged with PE-conjugated anti-CD46 antibodies. The particles localizes to the liver, spleen, and tumor.

FIG. 4 is a bar graph plotting the percent decrease in Kupffer cells at the indicated day post injection for mice treated with HA-Clod (hydroxyapatite-clodronate).

FIG. 5 is a bar graph plotting the percent ID for the indicated tissues from mice treated with either HA or HA-Clod.

FIG. 6A contains histogram plots of data obtained from flow cytometric analyses of SKOV3 and Raji cells that were incubated with PE-conjugated anti-Her2 antibodies (Her2-pe), hydroxyapatite particles (HAP) loaded with PE-conjugated anti-Her 2 antibodies (HAP-Her2-pe), PE-conjugated anti-CD20 antibodies (CD20-pe), or HAP loaded with PE-conjugated anti-CD20 antibodies (HAP-CD20-pe). FIG. 6B contains fluorescence photomicrographs obtained by confocal microscopy of SKOV3 cells incubated with HAP loaded with protein G and PE-conjugated anti-CD46 antibodies or PE-conjugated anti-CD20 antibodies.

FIG. 7 contains bioluminescent images monitoring tumor size over time in mice treated with hydroxyapatite particles (HAP), Sm-153-EDTMP (Sm), Sm-153-hydroxyapatite particles (Sm-HAP), or a combination of Sm-HAP and two doses of PS-341 (Sm-HAP+PS-341).

FIG. 8A contains chemical structures of phosphonates. A general chemical structure of a biphosphonate is presented. R1 and R2 are two side chains that can contribute to a difference in bioreactivity among biphosphonates. Structures of clodronate (R1=R2=Cl) and methylene diphosphonate (R1=R2=H) also are presented. FIG. 8B contains a schematic representation of an interaction between phosphonates and hydroxyapatite.

FIG. 9A contains X-ray diffraction graphs of synthesized particles confirming hydroxyapatite as the dominant phase. FIG. 9B contains transmission electron micrographs of synthesized particles. FIG. 9C contains a graph plotting particle size versus synthesis temperature, which shows that hydroxyapatite particle size increases with increase in synthesis temperature. The sizes of hydroxyapatite particles were measured from TEM micrographs (200 particles measured per size) and by dynamic light scattering (hydrodynamic diameter). Scale bar, 100 nm. FIG. 9D contains a graph plotting specific surface area versus synthesis temperature, which shows the relation between specific surface area of the hydroxyapatite particles with synthesis temperature.

FIG. 10A contains a graph plotting labeling efficiency versus particle size for binding of 100 μCi Tc-99m-MDP to 500 μg of hydroxyapatite particles. FIG. 10B contains a graph plotting the maximum amount of Tc99m-MDP bound per mg of hydroxyapatite particles versus input activity for the indicated particle sizes. FIG. 10C contains a graph plotting labeling efficiency (expressed as a percentage of input Tc99m-MDP bound per mg of HAP) versus input activity. Error bars represent standard deviation (mean±SD, n=3).

FIG. 11 contains gamma camera images showing the biodistribution of HAP-MDP-Tc99m in mice three hours after intravenous injections of (A) free Tc-99m, (B) Tc-99m-MDP, or (C and D) HAP-MDP-Tc-99m. Planar images (A-C) were acquired for five minutes or (D) using SPECT-CT. The CT and SPECT images were fused using the AMIRA software to generate a 3-D image. Delivery of Tc99m-MDP through hydroxyapatite particles has redirected the radionuclide from the skeleton (B) to the liver (C, D).

FIG. 12A contains a graph plotting percentage of injected dose per mL of blood versus time post-injection of HAP-MDP-Tc99m. Regardless of size, HAP-MDP-Tc99m was rapidly cleared from the bloodstream within 30 minutes post-injection. Each value represents the percentage of injected dose at different time points up to two hours. FIG. 12B contains a graph plotting percentage of injected dose versus organ for the indicated hydroxyapatite particle sizes. Tissue concentrations of radioactivity at two hours post-injection showed that HAP-MDP-Tc99m accumulated primarily in the liver, followed by the spleen. Results (mean±SD, n=3) have been corrected for background radiation and are expressed as % injected dose corrected for background radiation.

FIG. 13 contains transmission electron micrographs (TEM) of liver sections harvested from mice that were injected intravenously with (A) 40 nm and (B) 200 nm HAP-MDP-Tc99m. Tissues were collected two hours after tail vein injection of hydroxyapatite particles. Vesicles in Kupffer cells lining the sinusoids were filled with hydroxyapatite particles. Scale bar, 2 μm. KC, Kupffer cell, H, hepatocyte. White boxes indicate regions magnified in (C) and (D). Scale bar, 500 nm. Arrows point to vesicles containing numerous HAP-MDP-Tc99m particles. FIGS. 13E and 13F contain transmission electron micrographs of 40 nm particles and 200 nm particles, respectively. Scale bar, 100 nm.

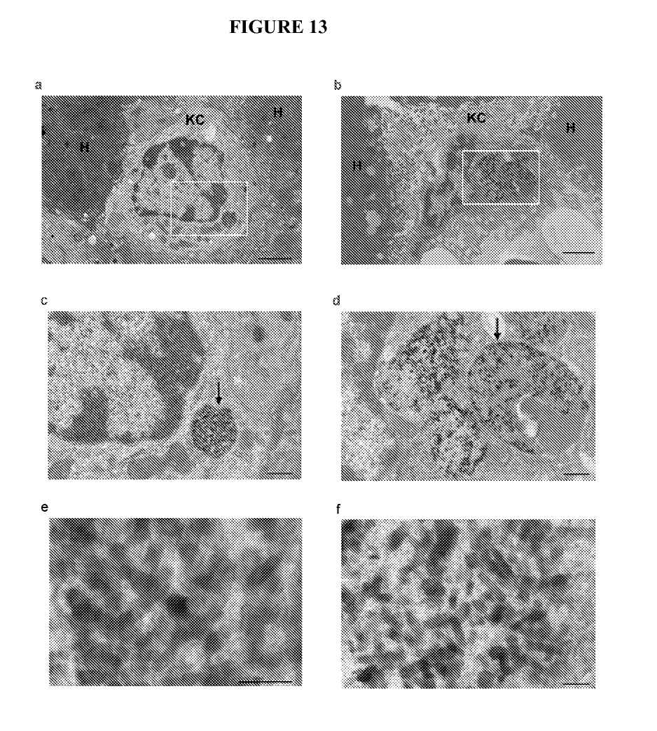

FIG. 14 contains photomicrographs of liver sections from mice administered clodronate loaded hydroxyapatite particles, or saline or hydroxyapatite particles as controls. The livers were harvested three days after administering the hydroxyapatite particles, sectioned, and immunostained with an anti-mouse CD68 antibody to detect the presence of Kupffer cells (blue).

### DETAILED DESCRIPTION

This document provides methods and materials related to hydroxyapatite particles. For example, this document provides hydroxyapatite particles, methods for making hydroxyapatite particles, and methods for using hydroxyapatite particles. The hydroxyapatite particles provided herein can have a radionuclide such as a radionuclide attached to a biphosphonate. In some cases, the hydroxyapatite particles provided herein can contain a binding molecule such as an antibody, receptor ligand, or nucleic acid. The binding molecule of such hydroxyapatite particles can be used to target the hydroxyapatite particles and radioactivity to a particular location within a mammal's body. For example, anti-CD46 antibodies can be attached to hydroxyapatite particles such that the hydroxyapatite particles are targeted to CD46 positive cells such as tumor cells.

This document also provides methods for making hydroxyapatite particles. For example, the hydroxyapatite particles provided herein can be made using a synthesis method that comprises adding biphosphonate-conjugated radionuclides during the synthesis of the hydroxyapatite particles. The resulting radioactive hydroxyapatite particles can be stable. In some case, binding molecules such as monoclonal antibodies can be added to the synthesis reaction such that the resulting radioactive hydroxyapatite particles can be targeted to, for example, specific cell surface receptors.

Hydroxyapatite particles can be any shape and can range in size from about 1 nm to about 5 μm in size (e.g., from about 2 nm to about 1 μm, from about 4 nm to about 750 nm, from about 20 nm to about 500 nm, from about 40 nm to about 400 nm, from about 50 nm to about 300 nm, or from about 60 nm to about 200 nm). Any method can be used to make hydroxyapatite particles. For example, wet chemistry (e.g., precipitation), hydrothermal, sol-gel, and hydrolysis of calcium phosphates techniques can be used to make hydroxyapatite particles (see, e.g., Wang M., Bioactive materials and processing. In: Shi D, editor, Biomaterials and tissue engineering. Berlin. Heidelberg: Springer; 2004. p. 1-82).

Hydroxyapatite particles can be synthesized by wet chemical precipitation at a temperature between about 1° C. and about 90° C. (e.g., 5° C., 10° C., 15° C., 20° C., 25° C., 30° C., 35° C., 40° C., 45° C., 50° C., 55° C., 60° C., 65° C., 70° C., 75° C., or 80° C.) using calcium nitrate tetrahydrate and ammonium dihydrogen phosphate. Chemicals can be synthesized or obtained from a commercial supplier (e.g., Sigma, St. Louis, Mo., USA). An aqueous solution can be prepared containing between 4 g and 5 g (e.g., about 4.1, 4.2, 4.3, 4.4, 4.5, 4.6, 4.7, 4.8, or 4.9 g) of calcium nitrate tetrahydrate (Ca(NO₃)₂.4H₂O) in about 80 mL of water. Another aqueous solution can be prepared containing between 1 g and 2 g (e.g., about 1.1, 1.2, 1.3, 1.4, 1.5, 1.6, 1.7, 1.8, or 1.9 g) of ammonium dihydrogen phosphate (NH₄H₂PO₄) in about 192 mL of water. Ammonium hydroxide (NH₄OH; e.g., 25% v/v) can be added to each solution to make the pH alkaline (e.g., a pH of about 8, 8.5, 9, 9.5, 10, 10.5, 10.6, 10.7, 10.8, 10.9, 11.0, 11.1, 11.2, 11.3, 11.4, or 11.5). NH₄H₂PO₄ can be added drop wise to the alkaline Ca(NO₃)₂ solution, and the solution can be stirred for about 0.5 hour or more (e.g., 1, 1.5, 2, 2.5, 2.6, 2.7, 2.8, or 2.9 hours or more) at a temperature between about 1° C. and 90° C. (e.g., 5° C., 10° C., 15° C., 20° C., 25° C., 30° C., 35° C., 40° C., 45° C., 50° C., 55° C., 60° C., 65° C., 70° C., 75° C., or 80° C.) to obtain particles. After stirring, the solution can be allowed to age for at least 2 hours (e.g., at least about 2.5, 3, or 3.5 hours). The precipitates can be washed multiple times (e.g., three times, four times, five times, or six times) with water (e.g., by resuspending the particles in water and spinning via centrifugation). Hydrothermal treatment can be performed on the synthesized hydroxyapatite. For example, about 15 mL of hydroxyapatite suspension (e.g., 5 mg/mL) can be introduced into hydrothermal bombs (e.g., Parr acid digestion bombs, model 4744) and kept in an oven at a temperature in the range of 150° C. to 250° C. (e.g., about 190° C., 195° C., 200° C., 205° C., or 210° C.) for about 24 hours. The thermally-treated hydroxyapatite suspensions can be freeze dried (e.g., using an Alpha 1-4 LSC, Christ, Germany) to obtain hydroxyapatite powders. The resulting particles can be characterized using, for example, X-ray diffraction (XRD), Dynamic Light Scattering (DLS), Transmission Electron Microscopy (TEM), or Brunauer-Emmett-Teller (BET). The size of the hydroxyapatite particles obtained can be varied depending on the temperature at which the solutions are added and the temperature during synthesis (Ferraz et al., J. Applied Biomaterials and Biomechanics, 2:74-80 (2004)). For example, an increase in particle size can be obtained by increasing the precipitation or synthesis temperature. In some cases, larger hydroxyapatite particles synthesized at higher temperatures can have a lower SSA to volume ratio.

Hydroxyapatite particles can be labeled with a radionuclide attached to a phosphonate. For example, hydroxyapatite particles can be labeled with Sm-153-EDTMP or Tc-99m MD. The particles can be labeled by incubating unlabeled hydroxyapatite particles with Sm-153-EDTMP or Tc-99m MDP. For example, between 0.5 mg and 1.5 mg (e.g., about 0.7, 0.8, 0.9, 1.0, 1.1, 1.2, or 1.3 mg) of hydroxyapatite particles can be incubated with about 50 μCi to about 500 μCi (e.g., about 50, 75, 100, 125, 150, 175, 200, 225, 250, 275, 300, 325, 350, 375, 400, 425, 450, 475, or 500 μCi) of Sm-153-EDTMP or Tc-99m MDP for about 5 minutes to about 120 minutes (e.g., 10, 15, 20, 30, 45, 60, 80, or 100 minutes) at room temperature. The reaction can be performed in Tris buffer (pH 7.2). The resulting labeled hydroxyapatite particles can be spun down and resuspended in 10 mM Tris buffer, pH 7.2. The amount of bound radioactivity can be measured using a dosimeter and can be calculated as a percentage of input radioactivity. The mixture can be sonicated prior to administration to a mammal.

Hydroxyapatite particles also can be labeled with a radionuclide attached to a phosphonate using a co-synthesis method. For example, hydroxyapatite particles can be labeled with Sm-153-EDTMP using a co-synthesis method. A solution of calcium nitrate tetrahydrate (Ca(NO₃)₂.4H₂O) can be made by combining between 4.0 g and 5.5 g (e.g., about 4.1, 4.2, 4.3, 4.4, 4.5, 4.6, 4.7, 4.8, 4.9, 5.0, 5.1, 5.2, 5.3, or 5.4 g) of calcium nitrate tetrahydrate (Ca(NO₃)₂.4H₂O) in about 80 mL of water. NH₄OH (e.g., 25% v/v) can be added to the calcium nitrate tetrahydrate solution to make the pH alkaline. A solution of ammonium dihydrogen phosphate (NH₄H₂PO₄) can be prepared by combining between 0.6 g and 2.5 g (e.g., about 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 g) of ammonium dihydrogen phosphate and 192 mL of de-ionized water. NH₄OH can be added to the ammonium dihydrogen phosphate solution to make the pH alkaline. In addition, between 40 mCi and 100 mCi (e.g., about 45, 50, 55, 60, 65, 70, 75, 80, 85, 90, 95, or 100 mCi) of Sm-153 EDTMP can be added to the ammonium dihydrogen phosphate solution. The ammonium dihydrogen phosphate solution can be added drop-wise to the calcium nitrate tetrahydrate solution, and the mixture can be stirred for about 30 minutes at room temperature. The mixture can be allowed to age for at least 2 hours (e.g., at least about 2.5, 3, or 3.5 hours). After aging, the precipitate can be washed repeatedly.

Hydroxyapatite particles can be loaded with one or more therapeutic agents. For example, hydroxyapatite particles can be loaded with a phosphonate. A phosphonate (e.g., a biphosphonate) can be added to hydroxyapatite particles in 10 mM Tris-Cl buffer (pH 7.2) and the mixture can be incubated at room temperature with continuous shaking. For example, between 10 milligrams and 40 milligrams (e.g., about 15, 20, 25, 30, or 35 milligrams) of clodronate (dichloromethylenediphosphonic acid disodium salt), a biphosphonate, can be mixed with about 1 mg of hydroxyapatite particles in 10 mM Tris-Cl buffer (pH 7.2). The clodronate can be dissolved in distilled water (e.g., at a concentration of about 200 mg/mL) prior to adding between 10 milligrams and 40 milligrams of the clodronate to the hydroxyapatite particles. The mixture can be incubated with continuous shaking at room temperature for about 30 minutes. After hydroxyapatite particle binding, free clodronate in the supernatant can be complexed with copper (II) ions (Cu²⁺) in nitric acid solution, and the concentration can be assayed by ultraviolet spectrophotometry as described elsewhere (Ostovic et al., Pharm Res., 10:470-472 (1993)). The amount of clodronate bound to hydroxyapatite particles can be calculated by subtracting free clodronate from input clodronate.

Hydroxyapatite particles can be attached (e.g., directly) to a binding molecule. For example, unlabeled hydroxyapatite particles can be designed to target CD20 by loading the particles with an anti-CD20 antibody. Between 50 μg and 200 μg (e.g., about 60, 70, 80, 90, 100, 110, 120, 130, 140, 150, 160, 170, 180, or 190 μg) of hydroxyapatite particles can be incubated with about 100 μg to 500 μg (e.g., about 150, 225, 250, 275, 300, 350, 400, or 450 μg) of anti-CD20 antibody for about 1 hour at 25° C. with agitation.

In some cases, radioactive hydroxyapatite particles (e.g., particles comprising a radionuclide attached to a phosphonate) can be attached to a binding molecule. For example, about 200 μCi of radioactive hydroxyapatite particles can be mixed with a binding molecule for about 1 hour at about 25° C. with agitation.

In some cases, protein G can be added to hydroxyapatite particles prior to adding antibodies in order to increase the number of antibodies bound per particle.

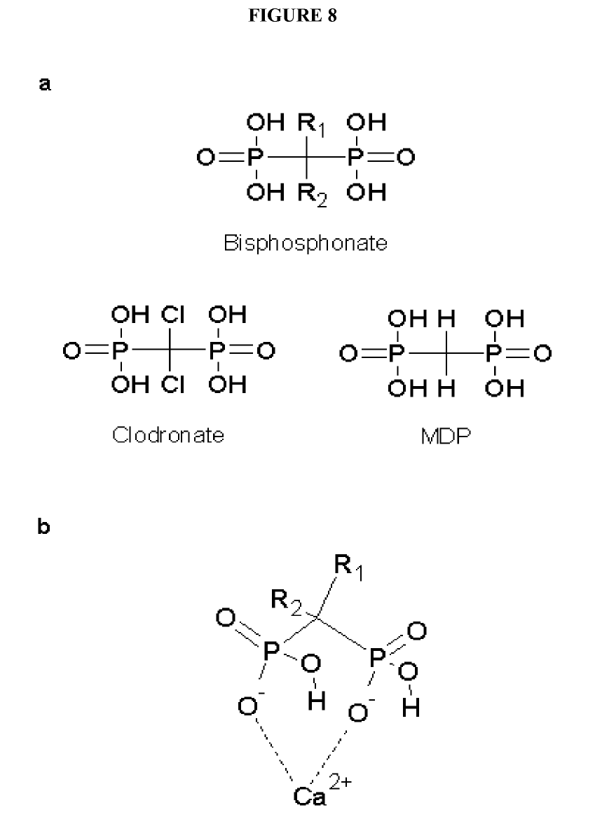

As described herein, phosphonates can be used to make hydroxyapatite particles. Examples of phosphonates (e.g., biphosphonates) that can be used to make hydroxyapatite particles include, without limitation, AEPn: 2-Aminoethylphosphonic acid; DMMP: Dimethyl methylphosphonate; HEDP: 1-Hydroxyethane (1,1-diylbisphosphonic acid); NTMP: Nitrilotris(methylenephosphonic acid); EDTMP: 1,2-Diaminoethanetetrakis (methylenephosphonic acid); DTPMP: Diethylenetriaminepentakis (methylenephosphonic acid); MDP: methylene diphosphonate; PBTC: Phosphonobutane-tricarboxylic acid, alendronate, ibandronate, zoledronate, incadronate, risedronate, EB-1053, neridronate, olpadronate, pamidronate, YH 529, tiludronate, etidronate, and clodronate. A general chemical structure of a biphosphonate is presented in FIG. 8A, along with chemical structures of clodronate and methylene diphosphonate. A schematic representation of an interaction between phosphonate and hydroxyapatite is presented in FIG. 8B. Clodronate and methylene diphosphonate (MDP) binding to hydroxyapatite can be bidentate. In this interaction, an oxygen atom from each phosphonate group can bind to a Ca²⁺ of hydroxyapatite.

As indicated herein, the hydroxyapatite particles provided herein can contain a radionuclide and/or a binding molecule. The binding molecule can be attached to the hydroxyapatite particles either directly or indirectly. For example, a binding molecule can be linked directly to the surface of a hydroxyapatite particle or indirectly through an intervening linker.

Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide functionalities. Examples of linkers include, without limitation, acetate and citrate linkers.

Any type of binding molecule can be attached to a hydroxyapatite particle. For example, a binding molecule can be a monoclonal antibody, folic acid, or a B12 vitamin. Tumor cells often overexpress surface antigens that allow them to be targeted by antibodies or small molecules. Some examples of surface molecules overexpressed on tumor cells that allow such targeting are CD20 (Rituxan) on lymphoma cells, Her-2/neu by Herceptin for breast cancer and ovarian cancer, and EGFR (by Cetuximab) and alpha-folate receptor for ovarian cancer (see, also, Table 1). As described herein, hydroxyapatite particles can be targeted to bind with high specificity to tumor cells expressing the targeted receptor via antibodies, folic acid, or a B12 vitamin.

TABLE 1
Cell Lines and the Surface Markers

| | Tumor Type | CD20 | Her2 | CD46 |
|---|---|---|---|---|
| Raji | B cell lymphoma | Positive | Negative | Positive |
| U87 | Glioblastoma | Negative | Negative | Positive |
| SKOV3 | Ovarian Cancer | Negative | Positive | Positive |
| KAS 6/1 | Multiple Myeloma | Negative | Negative | Positive |

In some cases, a therapeutic agent can be attached to a hydroxyapatite particle. Examples of a therapeutic agents include, without limitation, anti-angiogenic agents, chemotherapeutic agents, anti-inflammatory agents, anti-bacterial agents, anti-fungal agents, growth factors, immunostimulatory agents, anti-cholinergic agents, insulin, and insulin analogs.

Any type of a chemotherapeutic agent can be linked to an hydroxyapatite particle, including for example, taxol, vinblastin, vincristine, acyclovir, tacrine, gemcitabine, paclitaxel, herceptin, methotrexate, cisplatin, bleomycin, doxorubicin, and cyclophosphamide. Any combinations of such chemotherapeutic agents can be used. Any method for preparing chemotherapeutic agents can be used, including those described elsewhere. In some cases, an hydroxyapatite particle can have two or more therapeutic agents linked to it. For example, hydroxyapatite particles can have both a chemotherapeutic agent and a targeting antibody.

Any of the compositions provided herein can be formulated to form a pharmaceutically acceptable composition adapted for human or animal patients. Pharmaceutically acceptable means that the composition can be administered to a patient or animal without unacceptable adverse effects. Pharmaceutically acceptable compositions include any pharmaceutically acceptable salt, ester, or other derivative that, upon administration, is capable of providing (directly or indirectly) a composition of the invention. Other derivatives are those that increase the bioavailability of the compositions when administered or which enhance delivery to a particular biological compartment.

Where necessary, the pharmaceutically acceptable compositions can include such ingredients as solubilizing agents, excipients, carriers, adjuvants, vehicles, preservatives, a local anesthetic, salts, flavorings, colorings, and the like. The ingredients may be supplied separately, e.g., in a kit, or mixed together in a unit dosage form. A kit can further include directions for administering the hydroxyapatite particles provided herein and/or accessory items such as needles or syringes, etc.

Hydroxyapatite particles provided herein can be administered to mammals (e.g., for targeted delivery of radiation or therapeutic agents). For example, hydroxyapatite particles loaded with a phosphonate labeled radionuclide and/or a chemotherapeutic agent can be administered to a mammal having cancer (e.g., liver cancer) to target the radiation and/or the chemotherapeutic agent to the affected tissue (e.g., liver). In some cases, hydroxyapatite particles loaded with a binding molecule and a radionuclide or a therapeutic agent can be administered to mammal to target the radiation or therapeutic agent to a particular tissue. In some cases, hydroxyapatite particles comprising a radionuclide, a therapeutic agent, and a binding agent can be administered to a mammal to target the radiation and the therapeutic agent to a tissue.

Hydroxyapatite particles can be administered systemically or locally (e.g., at the site of a tumor). In addition, hydroxyapatite particles can be administered in amounts and for periods of time that can vary depending on the nature and severity of the condition being treated and the overall condition of the mammal. For example, hydroxyapatite particles can be administered to a mammal having cancer under conditions that reduce the progression rate of the cancer. In some cases, hydroxyapatite particles can be administered to a mammal having cancer to reduce the progression rate of the cancer by 5, 10, 25, 50, 75, 100, or more percent. For example, the progression rate can be reduced such that no additional cancer progression is detected. Any appropriate method can be used to determine whether or not the progression rate of cancer is reduced. For example, the progression rate of cancer can be assessed by imaging tissue at different time points and determining the amount of cancer cells present. The amounts of cancer cells determined within tissue at different times can be compared to determine the progression rate. After treatment with hydroxyapatite particles provided herein, the progression rate can be determined over another time interval. In some cases, the stage of cancer after treatment can be determined and compared to the stage before treatment to determine whether or not the progression rate was reduced. In some cases, reductions in cancer progression rates can be assessed using histological, biochemical, immunological, or clinical techniques. For example, histological techniques can be used to determine whether or not a tumor expanded into a particular tissue.

Hydroxyapatite particles provided herein also can be used to selectively deplete Kupffer cells in a mammal in order to minimize phagocytic uptake of systemically administered particles or viral vectors. For example, hydroxyapatite particles can be used to deliver a phosphonate (e.g., clodronate) to Kupffer cells in a mammal to mediate Kupffer cell killing. Hydroxyapatite particles comprising a phosphonate can be administered to a mammal systemically (e.g., via intravenous injection). Any appropriate method can be used to determine whether or not the number of Kupffer cells in the liver of the mammal is reduced. For example, a liver biopsy specimen can be obtained before and after administration of hydroxyapatite particles comprising a phosphonate. The biopsy specimens can be stained with an antibody directed against a marker for Kupffer cells (e.g., CD68), and the two stained biopsy specimens can be compared to determine whether or not administration of the hydroxyapatite particles reduced the number of Kupffer cells. Selectively killing Kupffer cells can reduce phagocytic uptake of particles or viral vectors targeted to other tissues which, in turn, can decrease the effective amount of particles or vectors.

In addition to being useful for delivery of therapeutic doses of isotopes (e.g., gamma- or beta-emitting) for radiation therapy, radiolabeled hydroxyapatite particles provided herein (e.g., hydroxyapatite particles labeled using gamma emitting radioisotopes) also can be administered to mammals to serve as imaging agents.

The invention will be further described in the following examples, which do not limit the scope of the invention described in the claims.

### EXAMPLES

### Example 1

### Synthesis of Non-Radioactive Particles

A solution of calcium nitrate tetrahydrate [Ca(NO₃)₂.4H₂O] was made by combining 4.72 g of calcium nitrate tetrahydrate and 80 mL de-ionized water. 3.6 mL of 25% v/v NH₄OH were added to the calcium nitrate tetrahydrate solution to make the pH alkaline. A solution of ammonium dihydrogen phosphate [NH₄H₂PO₄] was prepared by combining 1.38 g of ammonium dihydrogen phosphate and 192 mL of de-ionized water. 85.2 mL of 25% v/v NH₄OH were added to the ammonium dihydrogen phosphate solution to make the pH alkaline. The ammonium dihydrogen phosphate solution was added drop-wise to the calcium nitrate tetrahydrate solution. The mixture was then stirred for 30 minutes at room temperature, and allowed to age for three hours. After aging, the precipitate was repeatedly (5×) washed by resuspending in water and spinning via centrifugation.

The resulting precipitate contained hydroxyapatite particles having diameters ranging from 40 nm to 200 nm as determined via dynamic light scattering. The size of HA nanoparticles can vary depending on the temperature at which the solutions are added and the temperature during synthesis (Ferraz et al., J. Applied Biomaterials and Biomechanics, 2:74-80 (2004)).

The unlabeled, hydroxyapatite particles were labeled with Tc-99m MDP via chemisorption. Briefly, 1 mg of hydroxyapatite particles was incubated with 200 μCi of Tc-99m MDP for 15 minutes at room temperature (25° C.). The resulting Tc99m-labeled hydroxyapatite particles were spun down and resuspended in 10 mM Tris buffer pH 7.2 (volume as desired, e.g. 150-200 μL for intravenous injection into mouse) and sonicated for one minute prior to being administered to mice.

The unlabeled, hydroxyapatite particles were incubated with Sm-153-EDTMP. Briefly, 1 mg of hydroxyapatite particles was incubated with 200 μCi of Sm-153-EDTMP for up to 2 hours at room temperature (25° C.). The resulting hydroxyapatite particles were spun down and resuspended in 10 mM Tris buffer pH 7.2. The amount of Sm-153 radioactivity in the hydroxyapatite particles was determined and found to be negligible.

The unlabeled, hydroxyapatite particles were designed to target CD20 by loading the particles with PE-conjugated anti-CD20 antibody. Briefly, 100 μg of hydroxyapatite particles were incubated with 250 μg of anti-CD20 Ab for 1 hour at 25° C. with continuous agitation on a rotator. The resulting hydroxyapatite particles containing labeled anti-CD20 Ab were used in flow cytometry binding experiments to show specific binding of Ab labeled HA with target cells. Binding of labeled HA to cells was also demonstrated using microscopy and biodistribution studies in mice.

### Example 2

### Synthesis of Radioactive Particles

Sm-153-hydroxyapatite particles (Sm-HAP) were generated using a co-synthesis method.

A solution of calcium nitrate tetrahydrate [Ca(NO₃)₂.4H₂O] was made by combining 4.72 g of calcium nitrate tetrahydrate and 80 mL de-ionized water. 3.6 mL of 25% v/v NH₄OH were added to the calcium nitrate tetrahydrate solution to make the pH alkaline. A solution of ammonium dihydrogen phosphate [NH₄H₂PO₄] was prepared by combining 1.38 g of ammonium dihydrogen phosphate and 192 mL of de-ionized water. 85.2 mL of 25% v/v NH₄OH were added to the ammonium dihydrogen phosphate solution to make the pH alkaline. In addition, 60 mCi of Sm-153 EDTMP were added to the ammonium dihydrogen phosphate solution.

The ammonium dihydrogen phosphate solution was added drop-wise to the calcium nitrate tetrahydrate solution. The mixture was then stirred for 30 minutes at room temperature, and allowed to age for three hours. After aging, the precipitate was repeatedly (five times) spun down via centrifugation and washed in water.

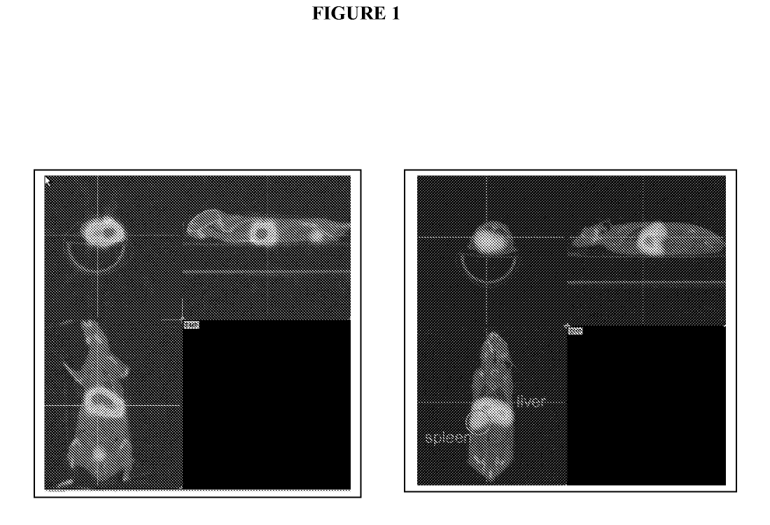

To determine the in vivo biodistribution of Sm-HAP, 200 μCi of Sm-HAP were administered intravenously to mice in 150 to 200 μL. The mice were imaged non-invasively using a microSPECT/CT machine. Significant uptake was observed in liver and spleen (FIG. 1). The spleen signal was much stronger when mice were given 500 μCi of Sm-HAP.

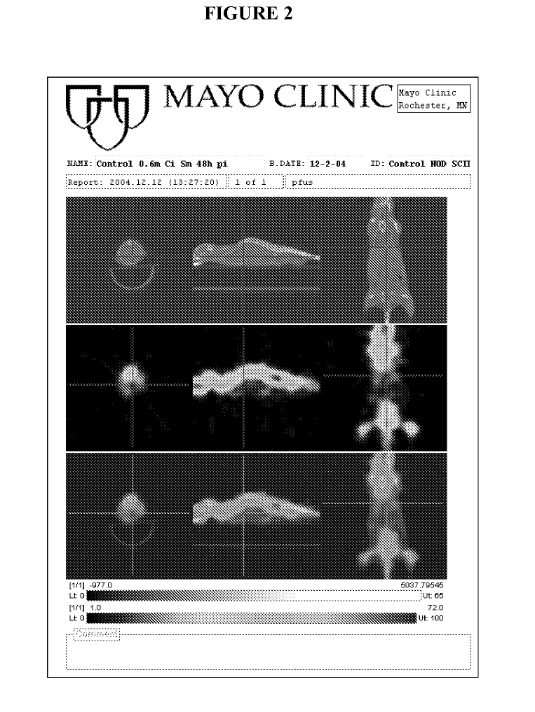

In contrast, infusion of free Sm-153-EDTMP not incorporated into an hydroxyapatite particle binds to the skeleton of the mouse due to the affinity of EDTMP for hydroxyapatite in the bone (FIG. 2).

### Example 3

### Synthesis of Radioactive Particles Containing a Targeting Molecule

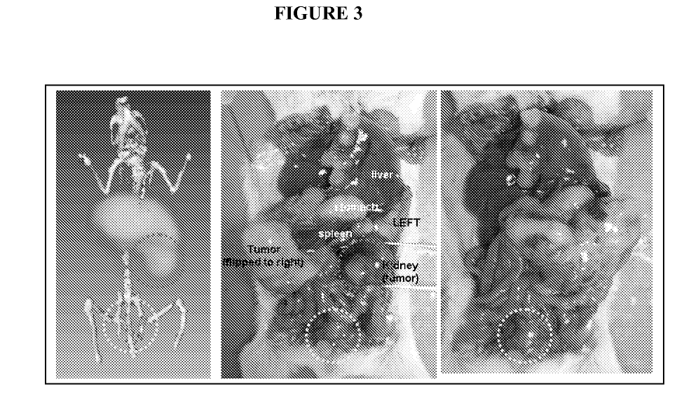

200 μCi of Sm-HAP were mixed with 2 μL, of PE-conjugated anti-CD46 Ab for 1 hour at 25° C. with continuous agitation on a rotator. The following experiment was performed to determine the in vivo biodistribution of CD46 targeted Sm-HAP. Sm-HAP containing PE-conjugated anti-human CD46 antibodies (500 μCi) were administered intravenously to scid mice bearing systemic KAS 6/1 tumors (advanced disease). The mice were imaged 30 minutes to 1 hour post infusion and euthanized later for necropsy. The particles localized to the liver, spleen, and tumor (FIG. 3).

High resolution analysis of fluorescent Sm-HAP in tissue and tumor slices was performed using fluorescence microscopy. Briefly, KAS 6/1 tumors were harvested from the mice and examined under fluorescence microscopy to locate the fluorescently tagged (through PE-conjugated anti-CD46 antibody) Sm-HAP. The fluorescent signal was detected in tumors, liver, and spleen. These results demonstrate that the particles remain stably tagged with the fluorescent signal even after infusion into the mice.

### Example 4

### Loading of Hydroxyapatite Particles with Clodronate (a Biphosphonate Drug) to Deplete Kupffer Cells in the Liver

Intracellularly, clodronate is metabolized to a toxic analog of adenosine triphosphate (ATP), β-γ-methylene, which ultimately leads to the induction of apoptosis in the target cells. Clodronate is a member of the biphosphonate family. The following experiment was performed to deliver clodronate via its binding to HA. The binding efficiency of clondronate to 40 nm HA was about 10%, with a final concentration of 2 mg clodronate/mg HA.

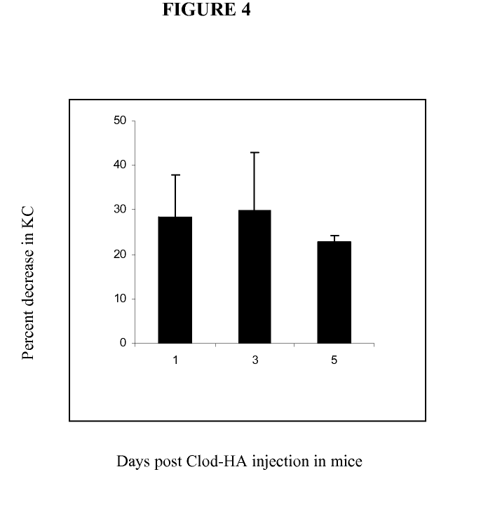

The degree and kinetics of depletion and repopulation of Kupffer cells (KCs) was analyzed following intravenous injection of HA-Clod (1 mg/10 g of body weight) in SCID mice. As a control, mice were given 1 mg of HA. At various time intervals, mice (n=3) were sacrificed, and the KC content was determined by immunostaining with anti-CD68 antibodies. The number of KCs was estimated using NIH Image J software. One day after injection of HA-Clod, the content of KCs was reduced by 30-40% (FIG. 4).

Maximum depletion was observed by day 3. Using HA-Clod, transient depletion of KCs in the liver was achieved with no histological signs of toxicity detected.

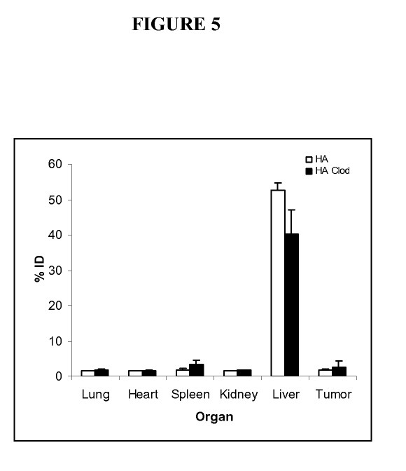

The effect of KC depletion on the biodistribution of HA was evaluated. Mice were implanted subcutaneously in the right flank with 5×10⁶ HT1080 human fibrosarcoma cells. When the tumors were well established, mice (n=4/group) were given HA-Clod intravenously, whereas a control group received HA. At day 3 post infusion, 200 μCi of HA-MDP-Tc99m were injected via tail vein. Pre-administration of HA-Clod resulted in up to a 13% decrease in liver uptake (FIG. 5), whereas pre-injection of HA did not change the liver uptake level compared to non-pre-treated mice. This ruled out the involvement of HA in influencing the subsequent HA biodistribution. KC depletion prior to injection of HA-MDP-Tc99m resulted in a higher tumor to liver ratio in comparison with control mice (3.3% vs. 6.4%). This improvement was likely due to the combined result of an absolute decrease of liver uptake and increase in tumor uptake. The increase in tumor accumulation could be attributed to higher concentration and longer blood circulation time of HA-MDP-Tc99m due to decreased uptake of particles by cells of the reticuloendothelial system (RES) of the liver.

### Example 5

### Targeting Hydroxyapatite Particles to Tumor Cells

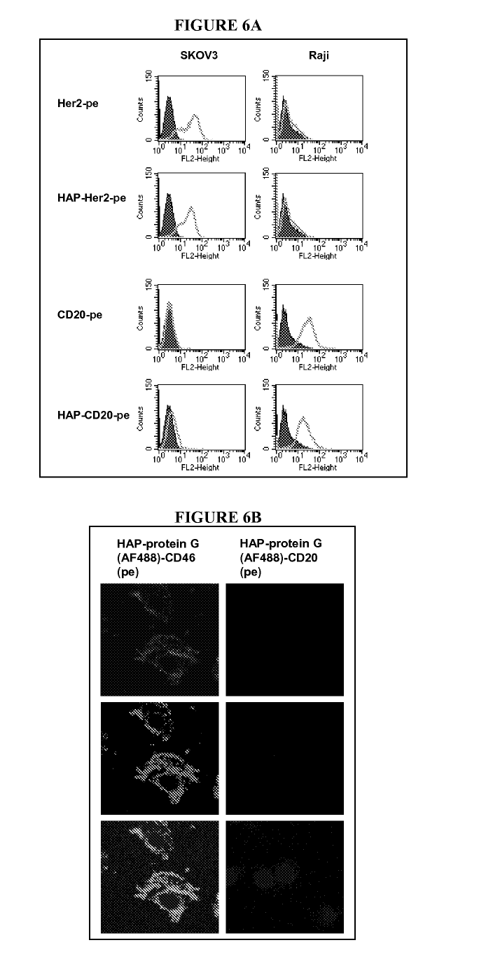

Hydroxyapatite particles were loaded with antibodies to target the particles to cell surface receptors. The antibody loaded particles were used in binding experiments to examine specific binding of antibody labeled HAP to target cells. One hundred μg of HAP were mixed with 2 L of PE-conjugated anti-CD20 antibody or PE-conjugated anti-Her2 antibody for one hour at 25° C. with continuous agitation on a rotator. The resulting HAP containing PE-conjugated anti-CD20 (HA-CD20-pe) antibody or PE-conjugated anti-Her2 (HA-Her2-pe) antibody were incubated with SKOV3 cells (human ovarian cancer cells; Her2 positive, CD20 negative) and Raji cells (human lymphoma cells; Her2 negative, CD20 positive) for one hour at 4° C. in the dark. After incubation, the cells were analyzed using a FACSCalibur Flow Cytometer. As controls, cells were incubated with free PE-conjugated antibody (Her2-pe, CD20-pe). Antibody conjugated HAP specifically bound to cells expressing the targeted receptor (FIG. 6A).

Specific binding of antibody conjugated HAP to target cells was also demonstrated using fluorescence microscopy. Briefly, 100 μg of HAP were loaded with Alexa Fluor 488-conjugated protein G, which has three IgG binding domains and thus increases the number of antibody bound per HAP. The HAP loaded with protein G were then bound with PE-conjugated CD46 antibody or with PE-conjugated CD20 antibody. The resulting HAP were then incubated with SKOV3 cells expressing surface CD46 polypeptides. Confocal microscopy analysis showed that HAP loaded with CD46 antibody bound to SKOV3 cells (FIG. 6B; red and green fluorescence was observed in the upper left panel and the middle left panel, respectively, and a composite of the fluorescent signals is shown in the lower left panel). In contrast, HAP loaded with CD20 antibody was not observed to bind to SKOV3 cells (FIG. 6B). These results showed that HAP can be targeted via antibodies to bind with high specificity to tumor cells expressing a targeted receptor.

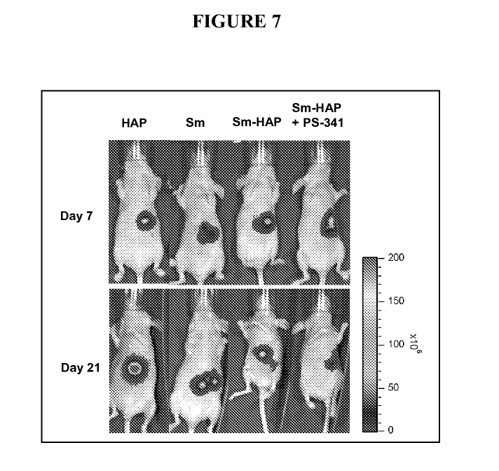

Experiments were performed to evaluate Sm-153-hydroxyapatite particles (Sm-HAP) alone or in combination with PS-341, a radiosensitizer drug, in a mouse liver cancer model. Nude mice were injected intrahepatically with Hep3B/Fluc, a human hepatocellular carcinoma cell line expressing Firefly luciferase. Tumor growth was determined using a Xenogen Bioluminescent IVIS 200 Imaging System. On day seven after tumor implantation, mice were injected intravenously via the tail vein with 200 μg HAP, 0.5 mCi free Sm-153-EDTMP, 0.5 mCi Sm-HAP alone, or 0.5 mCi Sm-HAP in combination with two intraperitoneal doses of 0.5 mg/kg PS-341, the first administered one day prior to Sm-HAP administration and the second administered one day after Sm-HAP administration. Tumors of mice treated with HAP or free Sm-153-EDTMP progressed, as evidenced by a brighter signal (FIG. 7), whereas mice treated with Sm-HAP has stable disease (FIG. 7). In contrast, tumors in mice treated with the combination therapy (Sm-HAP and two doses of PS-341) regressed, as evidenced by a decrease in the signal (FIG. 7).

### Example 6

### Using Hydroxyapatite Particles for Systemic Delivery of Radioisotopes and Drugs

Synthesis and characterization of hydroxyapatite particles: Hydroxyapatite particles were synthesized by wet chemical precipitation at various temperatures (5° C., 25° C., and 80° C.) using calcium nitrate tetrahydrate and ammonium dihydrogen phosphate. Chemicals were purchased from Sigma (St. Louis, Mo., USA). Aqueous solutions of calcium nitrate tetrahydrate (Ca(NO₃)₂.4H₂O; 4.704 g in 80 mL of water) and ammonium dihydrogen phosphate (NH₄H₂PO₄; 1.375 g in 192 mL of water) were prepared separately. Ammonium hydroxide (NH₄OH; 25% v/v) was pre-added to the solutions to raise the pH to 11 prior to the precipitation. NH₄H₂PO₄ was added drop wise to the alkaline Ca(NO₃)₂ solution and stirred continuously for three hours at 5° C., 25° C., and 80° C. to obtain particles of different sizes. The precipitates were washed five times with water. Hydrothermal treatment was performed on the synthesized hydroxyapatite, where 15 mL (5 mg/mL) of hydroxyapatite suspension was introduced into hydrothermal bombs (Parr acid digestion bombs, model 4744) and kept in a 200° C. oven for 24 hours. The thermally-treated hydroxyapatite suspensions were then freeze dried (Alpha 1-4 LSC, Christ, Germany) to obtain the hydroxyapatite powders. The resultant particles were characterized with X-ray diffraction (XRD), and particle sizes and specific surface areas (SSA) were measured using Dynamic Light Scattering (DLS), Transmission Electron Microscopy (TEM), and Brunauer-Emmett-Teller (BET).

X-ray Diffraction (XRD): The crystalline phases of the samples were determined using XRD. XRD was performed using a Shimadzu XRD-6000 Standard X-ray diffractometer employing CuK α radiation (λ=1.5406 Å) at 50 kV and 50 mA. Data were collected over the 20 range from 10-90° with a step size of 0.05° and scan rate of 5° min ⁻¹.

Transmission Electron Microscopy (TEM): Particle size was characterized using a JOEL 2010 TEM (2010 TEM, JOEL, Japan). Samples were prepared by mixing a small quantity of hydroxyapatite powder in ethanol followed by 10 minutes of ultrasonic treatment. A carbon coated copper grid was used to collect the samples from the solution and the images were captured via a built-in camera. Particle sizes were measured from the TEM micrographs on 200 particles, using the SPOT Basic software. Measurements were made on the longer particle dimension corresponding to the main c-axis of the crystal structure.

Dynamic Light Scattering (DLS): Dynamic light scattering (ZetaPlus, Brookhaven, USA) was used for hydrodynamic size measurements of the colloidal hydroxyapatite particles. The colloids were first prepared by dispersing 5 mg of particles in 0.1 wt % sodium hexametaphosphate solution, using ultrasound for 10 minutes. All measurements were performed at 25° C. at a measurement angle of 90°.

BET: Specific surface areas of the samples were estimated by a BET method using nitrogen as the absorption gas at 77 K (ASAP2000, Micromeritics, USA). The samples were degassed under vacuum at 200° C. overnight before analysis.

Radioisotope Tc-99m-MDP and clodronate loading on hydroxyapatite particles: Experiments were performed to determine the optimal amount of radioactivity that can be loaded on hydroxyapatite particles (HAPs) and to determine the optimal incubation time. In general, biphosphonate was added to hydroxyapatite particles in 10 mM Tris-Cl buffer (pH 7.2) and the mixture was incubated at room temperature with continuous shaking. To determine the optimal conditions for loading of Tc-99m-MDP to hydroxyapatite particles, the mixture was centrifuged and the amount of radioactivity associated with hydroxyapatite particles was measured at different time intervals (1, 3, 5, 10, and 15 minutes). Increasing amounts of Tc-99m-MDP (50, 100, 200, 300, 400, and 500 μCi) were added to 1 mg hydroxyapatite particles of 40, 100, and 200 nm. At the end of the incubation, the amount of bound radioactivity was measured using a well-type dosimeter and was calculated as a percentage of input Tc-99m-MDP. Labeling efficiency was calculated using the following equation.

$$Labeling Efficiency ( \% ) = \frac{Activity of HAP \text{-} MDP \text{-} Tc99 m}{Input Activity} \times 100$$

Clodronate (dichloromethylenediphosphonic acid disodium salt; Sigma, St. Louis, Mo.) was dissolved in distilled water (200 mg/mL) and 20 mg was added to 1 mg of hydroxyapatite particles. The mixture was incubated for 30 minutes. After hydroxyapatite particle binding, free clodronate in the supernatant was complexed with copper (II) ions (Cu²) in nitric acid solution and the concentration was assayed by ultraviolet spectrophotometry as described elsewhere (Ostovic et al., Pharm Res., 10:470-472 (1993)). The amount of clodronate bound to hydroxyapatite particles was calculated by subtracting free clodronate from input clodronate.

In vivo studies: Female athymic mice were purchased from Taconic Laboratory (Germantown, N.Y.) and allowed to acclimatize for one week before starting the experiments. Biodistribution of hydroxyapatite particles was studied in six week old female ICR outbred mice. For gamma-imaging studies, 100 μCi of HAP-MDP-Tc99m was administered through the tail vein in 200 μL Tris buffer. As controls, the same amounts of free Tc99m and free Tc99m-MDP were administered. At three hours post-infusion, the animals were anesthetized by intramuscular injection of ketamine/xylazine and imaged using a microSPECT-CT instrument (X-SPECT, Gamma Medica, CA). To study the role of particle size in hydroxyapatite particle biodistribution and blood clearance, mice were given intravenous injections of 100 μCi HAP-MDP-Tc99m. One hundred microliters of blood were collected retro-orbitally at various time intervals for up to two hours and the amount of radioactivity was measured. Two hours after infusion with HAP-MDP-Tc99m, mice were sacrificed by cervical dislocation and major organs were harvested. The radioactivity in each organ was measured using a well-type gamma spectrometer and was expressed as a percentage of injected dose per organ.

TEM and immunohistochemical staining of liver tissues: Samples of freeze dried hydroxyapatite particles were prepared in ethanol followed by 10 minutes of ultrasonic treatment. The particles were observed for their shape and size on TEM.

Particle sizes were measured from the TEM micrographs on 200 particles using the SPOT Basic software. Livers were harvested from mice injected with HAP-MDP-Tc99m, and the livers were fixed in Trumps fixative. Liver sections were visualized under a Philips CM10 Electron Microscope at the Electron Microscopy Core Facility at Mayo Foundation. For the immunohistochemical studies, liver samples were snap frozen and embedded in Optimal-Cutting-Temperature medium. The tissues were sectioned (5 μm) and fixed in −20° C. cooled acetone for three minutes. Immunohistochemical staining for Kupffer cells was performed. Briefly, tissues were permeabilized using 0.01% Triton-X in PBS for 10 minutes and washed three times in PBS. Endogenous peroxidase activity was quenched using 0.03% H₂O₂ in PBS for 30 minutes. The tissues were then washed and incubated with biotinylated rabbit anti-mouse CD68 (Serotec, Raleigh, N.C.) antibody (1:200 dilution) for one hour at room temperature, after which streptavidin was added and horse-radish peroxidase activity was detected using a substrate kit according to the manufacturer's instructions (Vector Laboratories, Burlingame, Calif.).

### Results

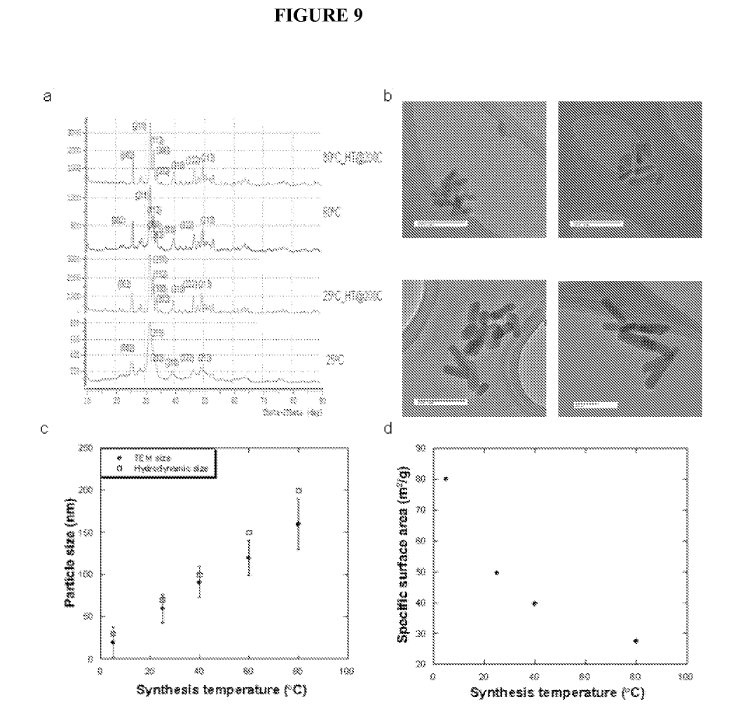

Hydroxyapatite particles were generated using wet chemical synthesis at various synthesis temperatures. X-ray diffraction of the precipitated particles confirmed hydroxyapatite as the predominant phase (FIG. 9A). The sizes of particles generated at the various temperatures were measured using transmission electron microscopy (FIG. 9B) and dynamic light scattering (FIG. 9C). The results indicated that there was an increase in particle size with increasing precipitation or synthesis temperature. The increase in size with temperature was in accordance with the theory of nucleation and growth. The specific surface areas (SSA) of the particles were measured using BET. The SSA decreased with increasing hydroxyapatite particle size (FIG. 9D), indicating that the larger hydroxyapatite particles synthesized at higher temperatures had a lower SSA to volume ratio.

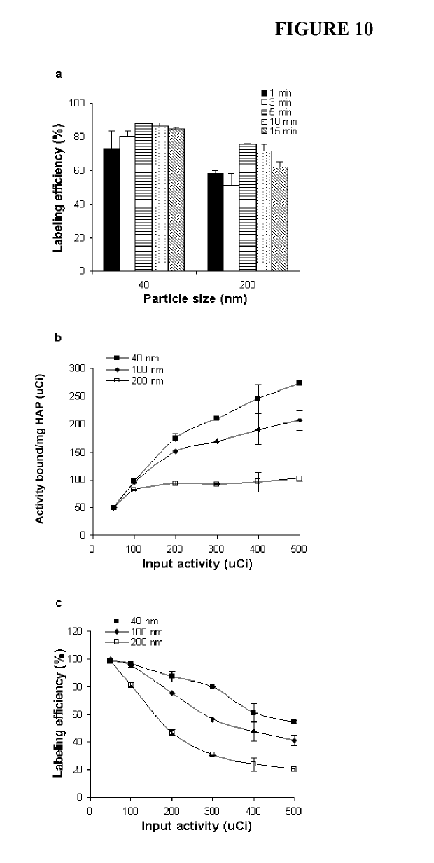

It was investigated whether radiolabeled hydroxyapatite particles could be generated to serve as imaging agents (using gamma emitting radioisotopes) or for delivery of therapeutic doses of beta-emitting isotopes for radiation therapy. To generate radiolabeled particles, the hydroxyapatite particles were loaded with a gamma-emitting probe, Tc-99m-methylene diphosphonate (Tc-99m-MDP). The optimal conditions required for maximum radiolabeling of the hydroxyapatite particles were established. Technetium-99m-MDP (100 μCi) was added to 1 mg of hydroxyapatite particles in Tris buffer (pH 7.2), and the resulting mixture was incubated with continuous shaking at room temperature. Binding of Tc99m-MDP to HAP was fast, reaching a maximum within 5 minutes (FIG. 10A). Prolonged incubation of up to 90 minutes did not increase the amount of radionuclide loaded. When increasing amounts of Tc-99m-MDP were added to hydroxyapatite particles of different sizes (40, 100, and 200 nm), the amount of radionuclide loaded was correlated with input dose (FIG. 10B) and the labeling efficiency was inversely correlated to particle size (FIG. 10C). Due to a higher SSA to volume ratio in smaller particles, the amount of Tc99m-MDP bound/mg HA was higher in 40 nm (274 μCi/mg) compared to 100 nm (206 μCi/mg) and 200 nm (102 μCi/mg) particles. This corresponded to a 1.3-fold and 2.7-fold increase, respectively (FIG. 10B).

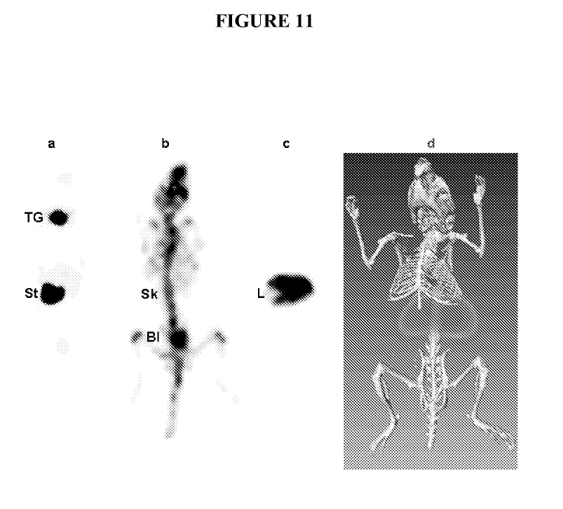

The biodistribution of Tc99m-MDP labeled hydroxyapatite particles after systemic injection in mice was monitored non-invasively via a small animal microSPECT-CT machine. One hundred μCi of HAP-Tc99m-MDP were injected intravenously via the tail vein into six week old female ICR outbred mice. Free Tc99m and free Tc99m-MDP were also injected into mice for comparison. Free Tc99m-MDP, a radionuclide that can be used in bone scintigraphy to diagnose osseous metastases in patients with cancer, localized rapidly to the skeleton (FIG. 11B), whereas free Tc-99m was taken up by the thyroid gland and stomach (FIG. 11A). In contrast, hydroxyapatite particle bound Tc-99m-MDP had a different biodistribution. Uptake of HAP-MDP-Tc99m in liver was observed shortly after intravenous administration (FIGS. 11C and 11D). These results indicate that delivery of Tc99m-MDP through hydroxyapatite particles redirected the localization of the radionuclide from the skeleton to the liver.

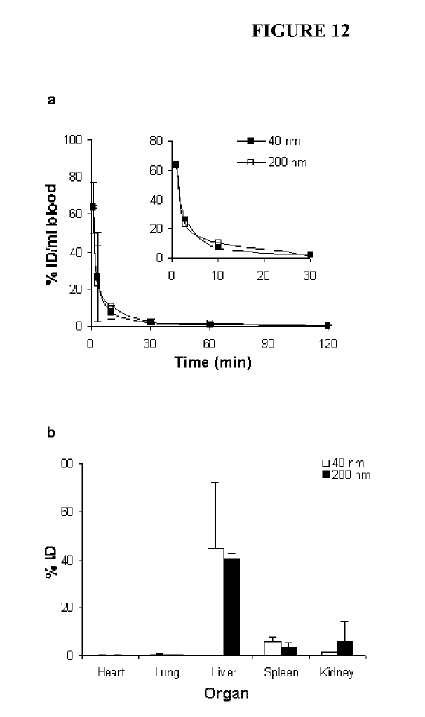

Size can play a role in the biodistribution of particles (Moghimi and Szebeni, Prog Lipid Res, 42:463-478 (2003); Senior, Crit. Rev Ther Drug Carrier Syst, 3:123-193 (1987); Pratten and Lloyd, Biochim Biophys Acta, 881:307-313 (1986)). The effect of size on the in vivo distribution of hydroxyapatite particles was studied using 40 nm and 200 nm particles. Mice were given intravenous injections of radiolabeled HAP-MDP-Tc99m. At various time intervals, the amount of radioactivity in a fixed volume of blood was measured. HAP-MDP-Tc99m was rapidly cleared from the blood circulation regardless of particle size (FIG. 12A). About 7-11% of the injected dose was still in circulation at 10 minutes post-injection and only 0.6-0.7% of the injected dose/mL was detected after 2 hours (FIG. 12A). Both 40 nm and 200 nm particles exhibited similar distribution profiles, with the largest portion of the administered HAP-MDP-Tc99m accumulating in the liver (FIG. 12B), in agreement with the imaging data. Uptake in the spleen was between 3-6% that of liver uptake, whereas the amount of HAP-MDP-Tc99m present in the kidney, lung, and heart was negligible. Overall, systemically administered HAP-MDP-Tc99m accumulated primarily in liver and this trafficking pattern did not appear to depend on particle size.

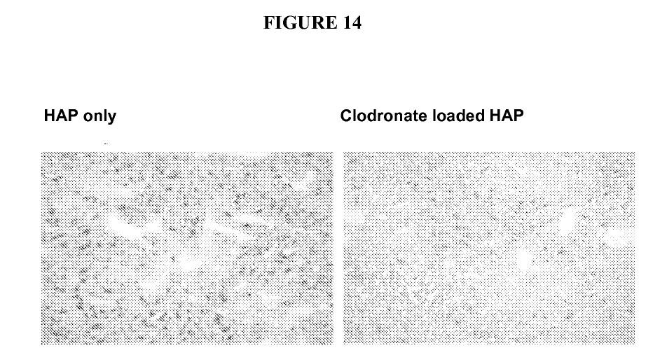

The amount of radioactivity found in the liver could be due to particle uptake by hepatocytes or by cells of the reticuloendothelial system. Liver sections of HAP-MDP-Tc99m-injected mice were, therefore, analyzed by transmission electron microscopy. Vesicles containing hydroxyapatite particles were seen in the majority of the Kupffer cells examined (FIGS. 14A-14D) and occasionally in vascular endothelial cells. No hydroxyapatite particles were detected in other cell types. Closer examination of the particle filled-vesicles at higher magnifications confirmed that the particles entrapped in the intracellular vesicles (FIGS. 14E and 14F) had the same size and shape as the respective particles prior to infusion into animals (FIG. 9B).

The feasibility of utilizing the HA-biphosphonate interaction for drug loading onto the particles also was tested. Twenty milligrams of clodronate, a biphosphonate, was mixed with 1 mg of hydroxyapatite particles (40 nm in size) in 10 mM Tris-Cl buffer (pH 7.2), and the mixture was incubated with continuous shaking at room temperature for 30 minutes. Clodronate could be loaded onto hydroxyapatite particles at 10% efficiency (2 mg clodronate/mg HAP; n=3). This is significantly higher than the reported 1% loading efficiency using liposomes (van Rooijen and van Kesteren-Hendrikx, Methods Enzymol, 373:3-16 (2003)). The clodronate that was loaded on hydroxyapatite particles remained biologically active. When given intravenously to mice, clodronate loaded hydroxyapatite particles resulted in selective killing of Kupffer cells. The liver sections of mice given drug loaded hydroxyapatite particles had less CD68 staining (a marker for Kupffer cells) compared to mice given hydroxyapatite particles alone (FIG. 15).

The results provided herein indicate that the phosphonates clodronate and Tc-99m-methylene-diphosphonate (Tc-99m-MDP) were efficiently loaded onto hydroxyapatite particles within 15 minutes. The biodistribution of the radiolabeled hydroxyapatite particles could be monitored non-invasively and regularly in the same animal using a microSPECT-CT machine. In addition, the results provided herein indicate that biphosphonate drugs or phosphonate labeled radionuclides can be used for hydroxyapatite particle loading, and the loaded particles can be useful for targeted delivery of radiation or drugs to the liver.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An article of manufacture comprising hydroxyapatite particles comprising a radionuclide attached to a biphosphonate, wherein the average diameter of said hydroxyapatite particles is between 40 nm and 200 nm.

2. The article of manufacture of claim 1, wherein said radionuclide comprises Sm-153, Tc-99m, ¹²³I, ¹⁸F, ¹³¹I, ¹¹¹In, ¹⁸⁸Re, ¹⁶⁶Ho, ⁹⁰Y, or ⁸²Rb.

3. The article of manufacture of claim 1, wherein said biphosphonate is ethylene diamine tetramethylene phosphoric acid or methylene diphosphonate.

4. The article of manufacture of claim 1, the average diameter of said hydroxyapatite particles is between 60 nm and 200 nm.

5. The article of manufacture of claim 1, wherein said hydroxyapatite particles comprise a binding molecule.

6. The article of manufacture of claim 5, wherein said binding molecule is an antibody.

7. The article of manufacture of claim 6, wherein said antibody is an anti-CD46 antibody, an anti-CD20 antibody, an anti-CD38 antibody, an anti-Her-2 antibody, an anti-EGFR antibody, an anti-α folate receptor antibody, an anti-MOV 18 antibody, or an anti-MOV19 antibody.

8. The article of manufacture of claim 5, wherein said binding molecule is a receptor ligand.

9. The article of manufacture of claim 1, wherein said particles comprise a nucleic acid molecule.

10. A method for making a radioactive hydroxyapatite particle, said method comprising synthesizing a hydroxyapatite particle in the presence of a radionuclide attached to a biphosphonate.

11. The method of claim 10, wherein said radionuclide comprises Sm-153, Tc-99m, ¹²³I, ¹⁸F, ¹³¹I, ¹¹¹In, ¹⁸⁸Re, ¹⁶⁶Ho, ⁹⁰Y, or ⁸²Rb.

12. The method of claim 10, wherein said biphosphonate is ethylene diamine tetramethylene phosphoric acid or methylene diphosphonate.

13. The method of claim 10, wherein the average diameter of said hydroxyapatite particles is between 40 nm and 200 nm.

14. The method of claim 10, wherein said hydroxyapatite particles comprise a binding molecule.

15. The method of claim 14, wherein said binding molecule is an antibody.

16. The method of claim 15, wherein said antibody is an anti-CD46 antibody, an anti-CD20 antibody, an anti-CD38 antibody, an anti-Her-2 antibody, an anti-EGFR antibody, an anti-α folate receptor antibody, an anti-MOV 18 antibody, or an anti-MOV 19 antibody.

17. The method of claim 14, wherein said binding molecule is a receptor ligand.

18. The method of claim 10, wherein said hydroxyapatite particle comprises a nucleic acid molecule.

19. A method for treating a mammal having cancer, said method comprising administering to said mammal hydroxyapatite particles comprising a radionuclide attached to a biphosphonate under conditions wherein the progression rate of said cancer is reduced.

20. The method of claim 19, wherein said mammal is a human.

21. The method of claim 19, wherein said cancer is selected from the group consisting of liver cancer, spleen cancer, and kidney cancer.

22. The method of claim 19, wherein said biphosphonate is ethylene diamine tetramethylene phosphoric acid or methylene diphosphonate.

23. The method of claim 19, wherein said radionuclide comprises Sm-153, Tc-99m, ¹²³I, ¹⁸F, ¹³¹I, ¹¹¹In, ¹⁸⁸Re, ¹⁶⁶Ho, ⁹⁰Y, or ⁸²Rb.

24. The method of claim 19, wherein said hydroxyapatite particles comprise a binding molecule.

25. The method of claim 19, wherein said hydroxyapatite particles comprise a therapeutic agent.

26. The method of claim 25, wherein said therapeutic agent is a chemotherapeutic agent or a phosphonate.

27. A method of depleting Kupffer cells in a mammal, said method comprising administering to said mammal hydroxyapatite particles comprising a phosphonate under conditions wherein the number of Kupffer cells in the liver of said mammal is reduced.

28. The method of claim 27, wherein said mammal is a human.

29. The method of claim 27, wherein said phosphonate is clodronate.
